(19) United States
(12) Patent Application Publication
(10) Pub. No.: US 2006/0251730 A1
(43) Pub. Date: Nov. 9, 2006

(54) Anti-microbial sanitary ware and method for making the same

(76) Inventor: Wen Lin Lo, Taichung (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/484,749

(22) Filed: Jul. 12, 2006

Related U.S. Application Data
(62) Division of application No. 10/797,818, filed on Mar. 10, 2004.

(51) Int. Cl. A61K 33/24; A61K 9/00

(52) U.S. Cl. 424/617; 424/400

(57) ABSTRACT

An anti-microbial sanitary ware includes a substrate, and an anti-microbial film formed on the substrate and including a protective layer and anti-microbial metal particles that are dispersed in the protective layer. The protective layer is made from a compound selected from the group consisting of metal nitrides and metal carbides. The anti-microbial metal particles are made from a metal selected from the group consisting of silver, zinc, and copper.

### BACKGROUND OF THE INVENTION

1. Field of the Invention

This invention relates to an anti-microbial sanitary ware and a method for making the same, more particularly to an anti-microbial sanitary ware with a metal particle-containing anti-microbial film and a method for making the same.

2. Description of the Related Art

Conventional sanitary wares, such as towel bars, robe hangers, faucets, shower heads, shelves, soap holders, paper holders, tumbler holders, door knobs etc., are normally coated with a protective film made from metal nitride or metal carbide so as to provide a smooth surface and resistance to corrosion and wearing for the sanitary wares. The protective film is formed on a substrate of the sanitary ware by sputtering techniques, such as unbalanced magnetron sputtering.

Since sanitary wares are constantly exposed to moisture-rich atmosphere, it is desirable to prevent or mitigate the growth of bacteria or fouling on the sanitary wares while maintaining the resistance to corrosion and wearing.

### SUMMARY OF THE INVENTION

Therefore, the object of the present invention is to provide an anti-microbial sanitary ware with an anti-microbial film that is capable of mitigating the growth of bacteria while maintaining the resistance to corrosion and wearing.

According to one aspect of the present invention, there is provided an anti-microbial sanitary ware that comprises: a substrate; and an anti-microbial film formed on the substrate and comprising a protective layer and anti-microbial metal particles that are dispersed in the protective layer. The protective layer is made from a compound selected from the group consisting of metal nitrides and metal carbides. The anti-microbial metal particles are made from a metal selected from the group consisting of silver, zinc, and copper.

According to another aspect of the present invention, there is provided a method for making an anti-microbial article. The method comprises the steps of: placing a substrate in a sputtering chamber in a sputter; and simultaneously sputtering a first metal target of a first metal and a second metal target of a second metal through closed-field unbalanced magnetron sputtering techniques, which form a continuously closed magnetic field around the substrate, so as to react the first metal into a metal compound which is subsequently deposited on the substrate to form a protective layer, and so as to generate metal particles of the second metal that are dispersed in the protective layer. The second metal is selected from the group consisting of silver, zinc, and copper. The metal compound is selected from the group consisting of metal nitrides and metal carbides.

### BRIEF DESCRIPTION OF THE DRAWINGS

In drawings which illustrate an embodiment of the invention,

FIG. 1 is a block diagram illustrating consecutive steps of the preferred embodiment of a method for making an anti-microbial sanitary ware according to the present invention; and

FIG. 2 is a cutaway view of the preferred embodiment of the anti-microbial sanitary ware formed according to the method of this invention.

### DETAILED DESCRIPTION OF THE PREFERRED EMBODIMENT

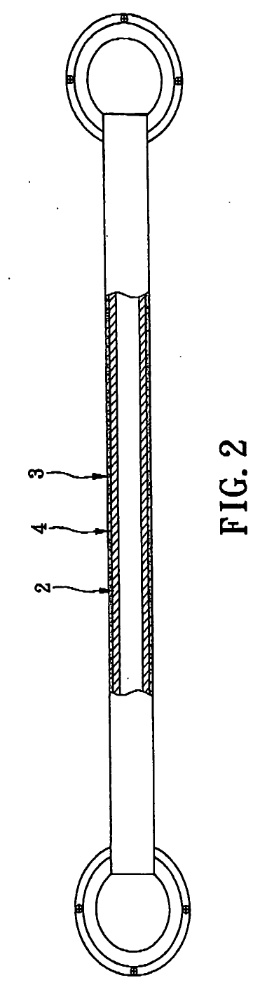

FIG. 2 illustrates the preferred embodiment of an anti-microbial sanitary ware, such as the aforesaid sanitary wares, according to the present invention. The anti-microbial sanitary ware includes: a substrate 2; and an anti-microbial film formed on the substrate 2 and comprising a protective layer 3 and anti-microbial metal particles 4 that are dispersed in the protective layer 3. The protective layer 3 is made from a metal compound selected from the group consisting of metal nitrides and metal carbides.

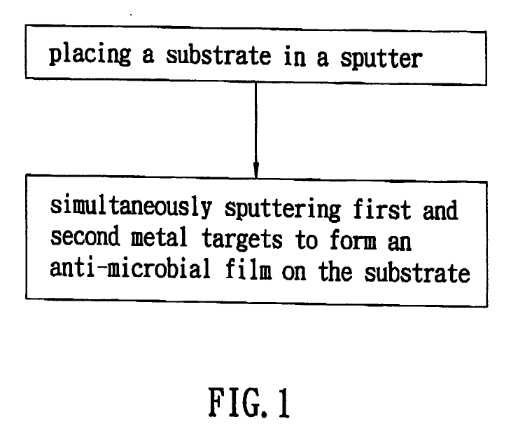

Referring to FIG. 1, in combination with FIG. 2, the method of forming the anti-microbial sanitary ware involves the use of closed-field unbalanced magnetron sputtering (CFUBMS) techniques, and includes the steps of: placing the substrate 3 in a sputter (not shown); and simultaneously sputtering a first metal target of a first metal and a second metal target of a second metal so as to react the first metal into the metal compound which is subsequently deposited on the substrate 2 to form the protective layer 3, and so as to generate the anti-microbial metal particles 4 of the second metal that are dispersed in the protective layer 3. The metal particles 4 thus formed have a particle size less than 100 nanometers.

Preferably, the second metal, which forms the anti-microbial metal particles 4, is selected from the group consisting of silver, zinc, and copper.

Preferably, the first metal is selected from the group consisting of zirconium, chromium, and titanium.

Preferably, the metal compound is metal nitride, and is selected from the group consisting of zirconium nitride, chromium nitride, and titanium nitride.

Preferably, the substrate is made from a material selected from the group consisting of copper alloy, zinc alloy, stainless steel, ceramics, and plastics.

The first metal sputtered from the first metal target reacts with a reactive gas, such as nitrogen, methane, or acetylene, in a sputtering chamber of the sputter (not shown) so as to form the metal compound.

Preferably, the sputtering of the first metal target is conducted at a voltage ranging from 20-50V, and a current ranging from 3.5-4.5 A, whereas the sputtering of the second metal target is conducted at a voltage less than 20V, and a current ranging from 0.3-0.5 A. Preferably, a negative biased voltage of greater than −80V is applied to the substrate 2.

Preferably, the sputtering is conducted at a temperature ranging from 80-180°, and a pressure ranging from 0.1-20 mTorr for 3-13 minutes.

The present invention will be described in more detail in the following Example.

### EXAMPLE 1

A substrate was subjected to milling and polishing, followed by cleaning and drying. The cleaned substrate was then prepared for deposition of the anti-microbial film using CFUBMS techniques, and was placed in a sputtering chamber which was filled with nitrogen and argon gas and which was provided with silver target and zirconium target. During the sputtering operation, a negative biased voltage of −50V was applied to the substrate, a positive biased voltage of less than 20V (with 0.4 A current) was applied to the silver target, and a positive biased voltage of 30V (with 4.0 A current) was applied to the zirconium target. The sputtering operation was conducted at a temperature of 180° C. and a pressure of 1.1 mTorr for 13 minutes so as to form an anti-microbial film (Ag—ZrN) on the substrate.

The thus formed anti-microbial sanitary ware was then subjected to anti-microbial activity tests in accordance with JIS Z 2801:2000 (Japanese Industry Standard).

The results show that the anti-microbial activity of the thus formed anti-microbial sanitary ware for escherichia coli is above 99.999%, and is above 99.9% for staphylococcus aureus and pseudomonas aeruginosa.

Since escape of the generated plasma in the sputtering chamber is effectively prevented by using CFUBMS techniques, which form a continuously closed magnetic field around the substrate, and since the generated plasma is confined in the continuously closed magnetic field, collision and ionization of molecules in the chamber are considerably increased, which result in an increase in the deposition of the material to be deposited on the substrate, which, in turn, results in an increase in the compactness and bulk density of the anti-microbial film deposited on the substrate. As a consequence, the resistance to corrosion and wearing of the anti-microbial sanitary ware of this invention is considerably enhanced.

The anti-microbial functions provided by the anti-microbial sanitary ware of this invention include one that interferes with the synthesis of the cell wall of bacteria, one that damages the cell membrane of bacteria, and one that interferes with the synthesis of nucleic acid. As a result of ionization, the metal particles 4 in the protective layer 3 of the anti-microbial sanitary ware of this invention tend to carry positive charges, which results in attraction of the bacteria, which, in turn, results in damage to the cell membrane and the enzyme system of the bacteria and the eventual destruction of the bacteria.

With the invention thus explained, it is apparent that various modifications and variations can be made without departing from the spirit of the present invention.

1. An anti-microbial sanitary ware comprising:
a substrate; and
an anti-microbial film formed on said substrate and comprising a protective layer and anti-microbial metal particles that are dispersed in said protective layer;
wherein said protective layer is made from a compound selected from the group consisting of metal nitrides and metal carbides; and
wherein said anti-microbial metal particles are made from a metal selected from the group consisting of silver, zinc, and copper.

2. The anti-microbial sanitary ware of claim 1, wherein said compound of said protective layer is metal nitride.

3. The anti-microbial sanitary ware of claim 2, wherein said compound is selected from the group consisting of zirconium nitride, chromium nitride, and titanium nitride.

4. The anti-microbial sanitary ware of claim 3, wherein said compound is zirconium nitride.

5. The anti-microbial sanitary ware of claim 4, wherein said substrate is made from a material selected from the group consisting of copper alloy, zinc alloy, stainless steel, ceramics, and plastics.

6. The anti-microbial sanitary ware of claim 5, wherein said substrate is made from copper alloy.

7-15. (canceled)
